(19) United States
(12) Patent Application Publication
(10) Pub. No.: US 2017/0087125 A1
(43) Pub. Date: Mar. 30, 2017

(54) FLAVONOID COMPOSITIONS FOR THE TREATMENT OF CANCER

(71) Applicant: Augusta University Research Institute, Inc., Augusta, GA (US)

(72) Inventor: Daqing Wu, Atlanta, GA (US)

(21) Appl. No.: 15/279,866

(22) Filed: Sep. 29, 2016

Related U.S. Application Data
(60) Provisional application No. 62/234,723, filed on Sep. 30, 2015.

(51) Int. Cl. A61K 31/353; A61K 45/06; A61K 47/44; A61K 9/00; A61K 9/48

(52) U.S. Cl. CPC A61K 31/353; A61K 9/0053; A61K 9/48; A61K 47/44; A61K 45/06

(57) ABSTRACT

Compositions containing luteolin, quercetin, and kaempferol are provided. The compositions are useful killing cancer cells and treating cancer. Exemplary cancers that can be treated include, but are not limited to prostate cancer and head and neck cancer.

### STATEMENT REGARDING FEDERALLY SPONSORED RESEARCH OR DEVELOPMENT

This invention was made with government support under grant number 1R41CA186498-01A1 awarded by the National Institutes of Health (NIH). The government has certain rights in the invention.

### FIELD OF THE INVENTION

The present invention relates to a formulation of bioactive flavonoids and its use in treating human cancers, including but not limiting to, prostate cancer and head and neck cancer.

### BACKGROUND OF THE INVENTION

Prostate cancer is the most common non-skin cancer in American men, with a lifetime risk for diagnosis of approximately 15.9%. Most cases of prostate cancer are low-risk (Gleason 6 or less, prostate-specific antigen, or PSA<10 ng/ml) and have a good prognosis with first-line treatment. Nonetheless, about 30% of patients harbor high-grade cancer and eventually progress, becoming metastatic and castration-resistant prostate cancer. Current therapies, including docetaxel, cabazitaxel, enzalutamide, abiraterone, denosumab and sipuleucel-T, can only extend the median survival by approximately 3 months. Most tumors relapse and become therapeutic-resistant, which is lethal with no cure. Further, these expensive treatments (usually ranging from $21,500˜$93,000 for a typical course of treatment) pose a huge burden on patients, their families and the healthcare system.

Numerous epidemiologic studies have indicated an important role of life styles, including diet, in cancer progression and therapeutic response. Significantly, the high prevalence and long latency period of low-risk prostate cancer provide a unique opportunity to control disease progression and improve the quality of life with dietary or nutraceutical approaches. Indeed, dietary management of prostate cancer is being actively pursued due to low dose-limiting toxicities and negligible side effects. Promising efficacy has been reported in certain trials.

Despite these encouraging clinical results, most trials with dietary supplements still suffer from small patient number, short treatment duration, and absence of proper placebo control. Importantly, the lack of standardized formulation and non-specific effects of these extract preparations make it difficult to validate and compare their clinical efficacy in various trials. Therefore, a nutraceutical formulation with defined composition and potent anti-cancer activity is highly desired, which may provide a safe, efficacious and cost-effective therapy for prostate cancer and other cancers.

It is an object of the invention to provide flavonoid compositions and methods of using them to treat cancer.

It is another object of the invention to provide flavonoid formulations for the treatment of cancer.

### SUMMARY OF THE INVENTION

Compositions containing luteolin, quercetin, and kaempferol at specific and reasonable ratios of the three flavonoids are provided. Luteolin, quercetin, and kaempferol are flavonoids and have the following structures:

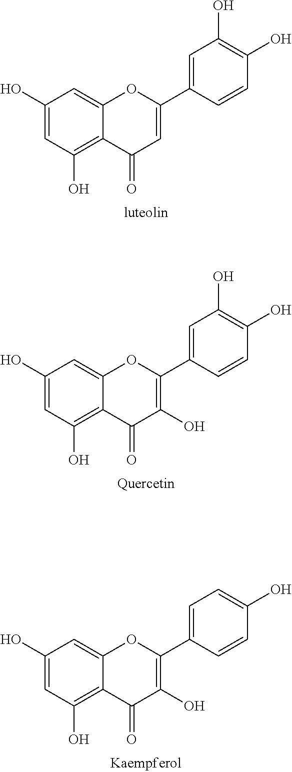 [SMILES: O=c1cc(-c2ccc(O)c(O)c2)oc2cc(O)cc(O)c12 ; O=c1c(O)c(-c2ccc(O)c(O)c2)oc2cc(O)cc(O)c12 ; O=c1c(O)c(-c2ccc(O)cc2)oc2cc(O)cc(O)c12]

In a preferred embodiment, the composition contains luteolin, quercetin, and kaempferol at a molar ratio of 1:1:2. Another embodiment provides a composition containing active ingredients consisting essentially of luteolin, quercetin, and kaempferol. The active ingredients may be formulated as a pharmaceutical composition in a pharmaceutically acceptable medium suitable for oral or parenteral administration.

Methods for treating cancer using the disclosed flavonoid compositions are also provided. One method of inhibiting cancer cells includes exposing the cancer cells to an inhibitory dose of the flavonoid compositions. The cells that are inhibited may be selected from metastatic and castration-resistant prostate cancer cells and head and neck cancer cells.

Another method provides treating a solid tumor in a mammalian subject, by administering to the subject, a therapeutically effective dose of the disclosed compositions, and repeating the administration at intervals of at least three times per week for a period of at least five weeks. A preferred subject is a human subject.

Still another method provides administering to a subject, either as a single regimen or combined with a second cancer treatment regimen selected from hormonal therapy, chemotherapy, and radiotherapy. Cancers that may be treated include, but not limited to, prostate cancer and head and neck cancer. Administration of the composition may enhance the efficacy of the second cancer treatment regimen.

The flavonoid composition can be administered on a daily basis at a daily dose between 1 to 200 mg/kg body weight, via oral, parenteral, intraperitoneal, intraveneous, subcutaneous routes, or by inhalation, by transdermal administration or trans-mucosal delivery.

### BRIEF DESCRIPTION OF THE DRAWINGS

FIGS. 1A-1B are line graphs of cell viability (%) versus amount of flavonoid composition (μg/ml). FIG. 1A shows cell viability of 3 prostate cancer cell lines (C4-2, CWR22Rv1). FIG. 1B shows cell viability of 5 head and neck cancer cell lines (SCC47, Fadu, TU686, PCI15A, JHUO22) following treatment for 72 hours, as determined by MTT or sulforhodamine B (SRB) assays.

FIG. 2 is a bar graph of percentage of apoptosis for amount of flavonoid composition (μg/ml) over 48 hours in a metastatic castration-resistant prostate cancer cell line (C4-2).

FIGS. 3A-3B are line graphs of relative cell viability versus Enzalutamide (μM) (FIG. 3A) or Docetaxel (FIG. 3B) at 0.0, 5.8 μg/ml, and 11.6 μg·ml of flavonoid composition for a metastatic castration-resistant prostate cancer cell line (C4-2) following the combined treatment with the flavonoid composition for 72 hours.

FIG. 4A is a bar graph showing the effect of the flavonoid composition on cell adhesion in a metastatic castration-resistant prostate cancer cell line (PC3). FIG. 4B is a photograph showing the effect of the flavonoid composition on cell migration of a metastatic castration-resistant prostate cancer cell line (PC3). FIG. 4C is bar graph showing the effect of the flavonoid composition on invasiveness of a metastatic castration-resistant prostate cancer cell line (PC3).

FIG. 5 shows the effect of flavonoid composition on gene expression in a metastatic castration-resistant prostate cancer cell line (C4-2).

FIG. 6A is a RT-PCR gel showing the effect of flavonoid composition and its individual active ingredients on the expression of androgen receptor (AR) (left. C: control; 1: Luteolin; 2: Quercetin; 3: Kaempferol). FIG. 6B is a bar graph of PSA mRNA Relative Quantity (dRn) for control of flavonoid composition in a metastatic castration-resistant prostate cancer cell line (C4-2). FIG. 6C is bar graph of PSA in supernatant (ng/ml) for control or flavonoid composition in a metastatic castration-resistant prostate cancer cell line (C4-2) by ELISA. FIG. 6D is a Western blot showing the effect of flavonoid composition and its individual active ingredients on the expression of androgen receptor (AR) (left. C: control; 1: Luteolin; 2: Quercetin; 3: Kaempferol).

FIGS. 7A-7D show the effect of the flavonoid compositions on the expression of indicated oncogenic signaling molecules in metastatic castration-resistant prostate cancer cell line (C4-2), as determined by Western blotting.

FIG. 8 is a schematic showing a proposed mechanism of action of flavonoid composition in inhibiting prostate cancer cells.

FIG. 9 shows a corn oil-based oral formulation of the flavonoids.

FIG. 10 is a line graph of body weight (g) versus treatment (day) of CD-1 mice following oral flavonoid composition administration at the doses of 200 mg/kg and 400 mg/kg for 16 days. Flavonoid composition was administered daily via oral gavage between Day 1 and Day 7, and switched to a schedule of three times per week between Day 8 and Day 16.

FIG. 11 is a line graph of tumor volume versus time (day) showing the effect of oral flavonoid composition on the subcutaneous growth of prostate cancer xenografts in athymic nude mice.

FIGS. 12A-12C are photographs showing bioluminescence imaging of prostate cancer xenografts in athymic nude mice following flavonoid composition administration.

FIG. 13 is a line graph of survival probability versus overall survival (day) showing the survival of nude mice following the administration with oral flavonoid composition.

FIG. 14A is a line graph of cell viability (%) versus cisplatin (μg/ml) showing the combined effect of flavonoid composition and cisplatin chemotherapy in a chemoradiation-resistant human head and neck cancer cell line PCI15A. FIG. 14B is a bar graph of survival fraction for PCI15A cells treated with 11.6 μg/ml flavonoid composition, radiation (2Gy), or both flavonoid composition and radiation in a chemoradiation-resistant human head and neck cancer cell line PCI15A.

FIG. 15A-15B are line graphs of cell viability (%) versus cisplatin (μg/ml) shows the combined effect of flavonoid composition and cisplatin in human head and neck cancer cell lines Fadu (FIG. 15A) and TU686 (FIG. 15B).

FIG. 16 is an autoradiograph showing the effect of flavonoid composition on head neck cancer cell signaling molecules.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “individual”, “host”, “subject”, and “patient” are used interchangeably herein, and refer to a mammal, including, but not limited to, murines, simians, humans, mammalian farm animals, mammalian sport animals, and mammalian pets.

As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

The tem “consisting essential of” limits the scope of a claim to the specified materials or steps “and those that do not materially affect the basic and novel characteristic(s)” of the claimed invention. In re Herz, 537 F.2d 549, 551-52, 190 USPQ 461, 463 (CCPA 1976).

### II. Flavonoid Compositions

Compositions containing luteolin, quercetin, and kaempferol are provided. In a preferred embodiment, the composition contains luteolin, quercetin, and kaempferol at a molar ratio of 1:1:2. Another embodiment provides a composition containing active ingredients consisting essentially of luteolin, quercetin, and kaempferol. The active ingredients may be formulated as a pharmaceutical composition in a pharmaceutically acceptable medium suitable for oral or parenteral administration.

In another embodiment, the composition contains luteolin, quercetin, and kaempferol at a molar ratio of 1:2:2; 2:1:2; 1:1:3. 1:2:3; 2:1:3; 3:1:2; 3:1;1; or 3:3:2.

The three active ingredients of are water-insoluble. To improve the in vivo absorption, stability and bioavailability, suitable aqueous and nonaqueous carriers that may be employed in the pharmaceutical compositions including water, ethanol, polyols, and suitable mixtures thereof, vegetable oils, such as corn oil, olive oil, and injectable organic esters. It will be appreciated that the flavonoids can be modified to increase water solubility for example by forming a salt of the flavonoid.

A. Luteolin

Luteolin, 3′,4′,5,7-tetrahydroxyflavone, is a common flavonoid that exists in many types of plants including fruits, vegetables, and medicinal herbs. Plants rich in luteolin have been used in Chinese traditional medicine for treating various diseases such as hypertension, inflammatory disorders, and cancer. Having multiple biological effects such as anti-inflammation, anti-allergy and anticancer, luteolin functions as either an antioxidant or a pro-oxidant biochemically. The structure of luteolin is shown below.

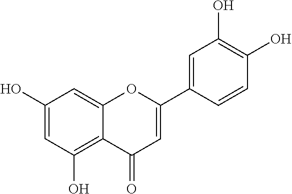 [SMILES: O=c1cc(-c2ccc(O)c(O)c2)oc2cc(O)cc(O)c12]

B. Quercetin

Quercetin, 3,3′,4′5,7-Penthydroxyflavone, is a flavonoid found in many plants and foods, such as red wine, onions, green tea, apples, berries, Ginkgo biloba, St. John's wort, American elder, and others. Buckwheat tea has a large amount of quercetin. The structure of quercetin is shown below.

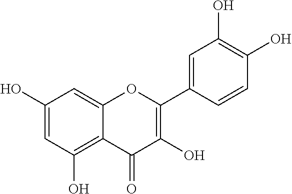 [SMILES: O=c1c(O)c(-c2ccc(O)c(O)c2)oc2cc(O)cc(O)c12]

C. Kaempferol

Kaempferol, (3,5,7-trihydroxy-2-(4-hydroxyphenyl)-4H-1-benzopyran-4-one), is a flavonoid found in many edible plants (e.g., tea, broccoli, cabbage, kale, beans, endive, leek, tomato, strawberries and grapes) and in plants or botanical products commonly used in traditional medicine (e.g., Ginkgo biloba, Tilia spp, Equisetum spp, Moringa oleifera, Sophora japonica and propolis). The structure of kaempferol is shown below.

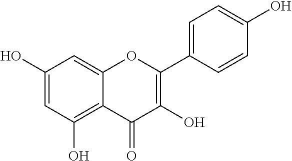 [SMILES: O=c1c(O)c(-c2ccc(O)cc2)oc2cc(O)cc(O)c12]

### III. Formulations

Formulations containing luteolin, quercetin and kaempferol can be made for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration. A preferred formulation is a pharmaceutical composition for oral administration.

The compositions can be formulated for immediate release, extended release, or modified release. A delayed release dosage form is one that releases a flavonoid at a time other than promptly after administration. An extended release dosage form is one that allows at least a twofold reduction in dosing frequency as compared to that drug presented as a conventional dosage form (e.g., as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

In one embodiment, formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the flavonoids. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman, et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et.al., (Media, Pa.: Williams and Wilkins, 1995) which provides information on carriers, materials, equipment and process for preparing tablets and capsules and delayed release dosage forms of tablets, capsules, and granules.

The composition can be administered to a subject with or without the aid of a delivery vehicle. Appropriate delivery vehicles for the compounds are known in the art and can be selected to suit the particular active agent. For example, in some embodiments, the flavonoids are incorporated into or encapsulated by, or bound to, a nanoparticle, microparticle, micelle, synthetic lipoprotein particle, or carbon nanotube. For example, the flavonoids can be incorporated into a vehicle such as polymeric microparticles which provide controlled release of the active agent(s). In some embodiments, release of the flavonoids is controlled by diffusion of the flavonoids out of the microparticles and/or degradation of the polymeric particles by hydrolysis and/or enzymatic degradation.

The compositions can include polymers. Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, all three flavonoids are incorporated into the same particles and are formulated for release at different times and/or over different time periods. For example, in some embodiments, one of the flavonoids is released entirely from the particles before release of the second or third flavonoid begins. In other embodiments, release of the first flavonoid begins followed by release of the second and third flavonoids before the all of the first flavonoid is released. In still other embodiments, both flavonoids are released at the same time over the same period of time or over different periods of time.

1. Formulations for Parenteral Administration

The compositions can be administered in an aqueous solution, by parenteral injection. The formulation may also be in the form of a suspension or emulsion. In general, pharmaceutical compositions are provided including effective amounts of the flavonoids and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Oral Immediate Release Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the flavonoid-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

3. Extended Release Dosage Forms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

4. Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Methods of Manufacturing

As will be appreciated by those skilled in the art and as described in the pertinent texts and literature, a number of methods are available for preparing flavonoid-containing tablets, beads, granules or particles that provide a variety of flavonoid release profiles. Such methods include, but are not limited to, the following: coating a flavonoid or flavonoid-containing composition with an appropriate coating material, typically although not necessarily incorporating a polymeric material, increasing particle size, placing the flavonoids within a matrix, and forming complexes of the flavonoids with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1995).

A preferred method for preparing extended release tablets is by compressing a flavonoid-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix flavonoid particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing flavonoid-containing beads involves dispersing or dissolving the flavonoid in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing flavonoid beads is by blending flavonoids with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### IV. Methods of Treatment

A. Methods of Treating Cancer

The disclosed flavonoid compositions can be used to treat cancer including tumors. Treatment that is administered in addition to a first therapeutic agent, for example the flavonoid compositions, to treat tumors is referred to as adjuvant therapy. Adjuvant treatment is given to augment the flavonoid composition treatment, such as surgery or radiation, to decrease the chance that the cancer will recur. This additional treatment can result in an amplification of the response due to the flavonoid compositions as evidenced by a more potent and/or prolonged response.

There are five main types of adjuvant therapy (note that some of these are also used as primary/monotherapy as well): 1.) Chemotherapy that uses drugs to kill cancer cells, either by preventing them from multiplying or by causing the cells to self-destruct, 2.) Hormone therapy to reduce hormone production and prevent the cancer from growing, 3.) Radiation therapy that uses high-powered rays to kill cancer cells, 4.) Immunotherapy that attempts to influence the body's own immune system to attack and eradicate any remaining cancer cells. Immunotherapy can either stimulate the body's own defenses (cancer vaccines) or supplement them (passive administration of antibodies or immune cells), or 5.) Targeted therapy that targets specific molecules present within cancer cells, leaving normal, healthy cells alone. For example, many cases of breast cancer are caused by tumors that produce too much of a protein called HER2. Trastuzumab (Herceptin®) is used as adjuvant therapy that targets HER2 positive tumors.

Typically adjuvant treatments are co-administered or given in conjunction with flavonoid composition treatments to induce multiple mechanisms and increase the chances of eradicating the tumor. Immunotherapy, and vaccines in particular, offer the unique advantages of inducing a sustained antitumor effect with exquisite specificity and with the ability to circumvent existing immune tolerance.

B7 costimulatory polypeptides, variants thereof and fusion proteins thereof and nucleic acids encoding the same may be useful in the induction or enhancement of an immune response to tumors when combined with the flavonoid compositions.

Malignant tumors which can be treated using the flavonoid compositions are classified herein according to the embryonic origin of the tissue from which the tumor is derived and are described below. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated in with the provided compositions and methods include, but are not limited to, the following: bladder, brain, breast, cervical, colo-rectal, esophageal, kidney, liver, lung, nasopharangeal, pancreatic, prostate, skin, stomach, uterine, head and neck and the like. Administration is not limited to the treatment of an existing tumor but can also be used to prevent or lower the risk of developing such diseases in an individual, i.e., for prophylactic use.

One method of inhibiting cancer cells includes exposing the cancer cells to an inhibitory dose of the flavonoid compositions. The cells that are inhibited may be selected from metastatic and castration-resistant prostate cancer cells and head and neck cancer cells.

Another method provides treating a solid tumor in a mammalian subject, by administering to the subject, a therapeutically effective dose of the disclosed compositions, and repeating the administration at intervals of at least three times per week for a period of at least five weeks. A preferred subject is a human subject.

Still another method provides administering to a subject, either as a single regimen or combined with a second cancer treatment regimen selected from hormonal therapy, chemotherapy, and radiotherapy. Cancers that may be treated include, but not limited to, prostate cancer and head and neck cancer. Administration of the composition may enhance the efficacy of the second cancer treatment regimen.

The flavonoid composition can be administered on a daily basis at a daily dose between 1 to 200 mg/kg body weight, via oral, parenteral, intraperitoneal, intraveneous, subcutaneous routes, or by inhalation, by transdermal administration or trans-mucosal delivery.

B. Combination Therapy

The disclosed compositions can be administered alone or in combination with one or more additional therapeutic agents. For example, the disclosed compositions can be administered with an antibody or antigen binding fragment thereof specific for a growth factor receptors or tumor specific antigens. Representative growth factors receptors include, but are not limited to, epidermal growth factor receptor (EGFR; HER1); c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cekl); fibroblast growth factor receptor 2 (Bek/Cek3/K-Sam); Fibroblast growth factor receptor 3; Fibroblast growth factor eceptor 4; nerve growth factor receptor (NGFR) (TrkA); BDNF receptor (TrkB); NT-3-receptor (TrkC); vascular endothelial growth factor receptor 1 (Flt1); vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Axl; RYK; DDR; and Tie.

Additional therapeutic agents include conventional cancer therapeutics such as chemotherapeutic agents, cytokines, chemokines, cryotherapy, hormone therapy, and radiation therapy. The majority of chemotherapeutic drugs can be divided into: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the new tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

### EXAMPLES

### Example 1

### Pharmaceutical Composition of Formula I

An example of Formula I is ProFine™, a composition containing pure, pharmaceutical-grade Luteolin, Quercetin and Kaempferol at a molar ratio of 1:1:2. To make a stock solution of ProFine™ at 100 mg/ml, 24.68 mg (86.23 μmol) Luteolin, 26.06 mg (86.23 μmol) Quercetin, and 49.35 mg (172.43 μmol) Kaempferol were added to 100% dimethyl sulfoxide (DMSO) to a final volume of 1.0 ml.

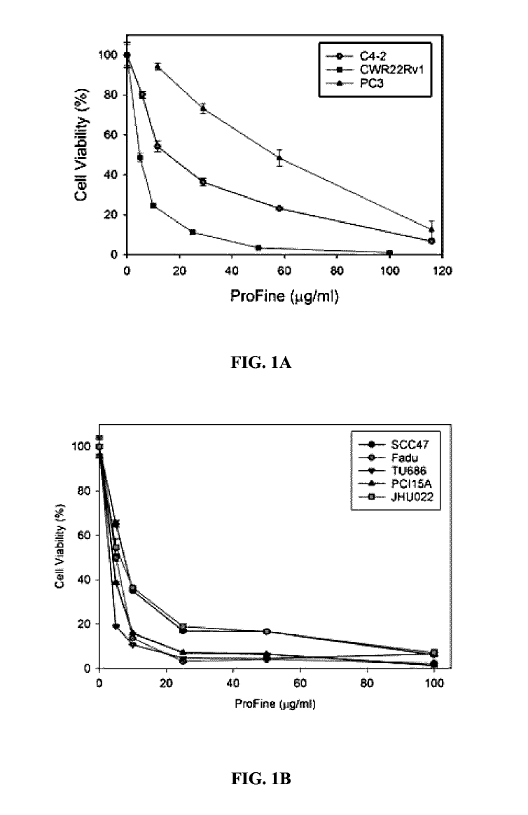

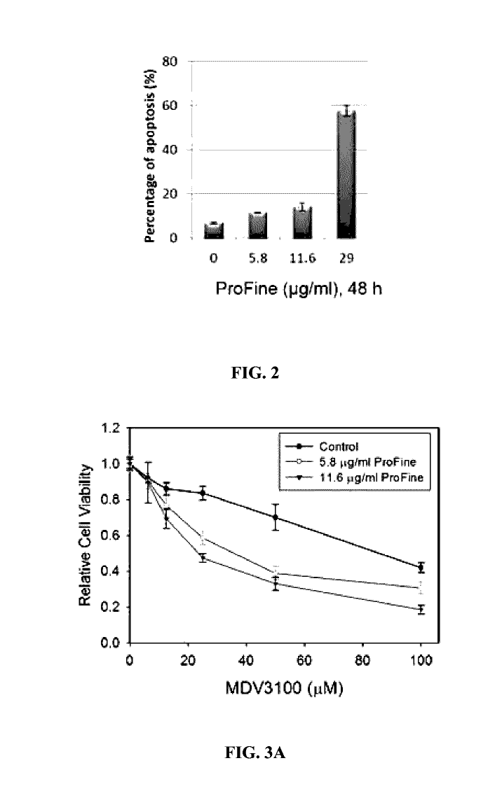

ProFine™ demonstrates potent anti-cancer activity in a wide range of human cancer cell lines, including prostate cancer and head and neck cancer cells (FIG. 1). Annexin V analysis shows ProFine™ effectively induces apoptosis in metastatic castration-resistant prostate cancer cells in a dose-dependent manner (FIG. 2).

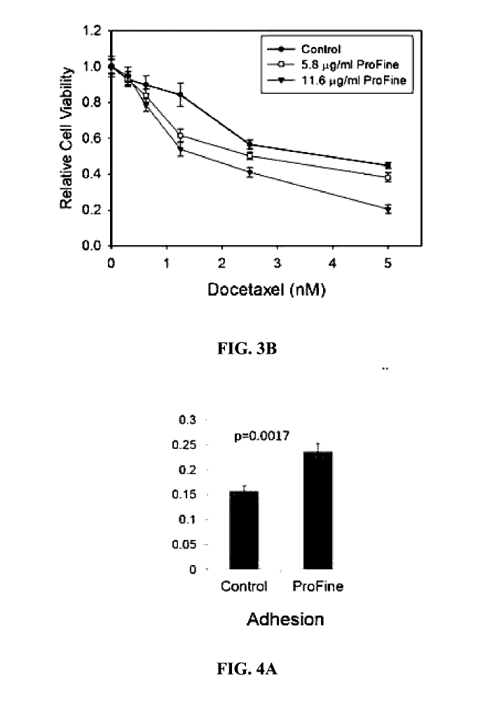

When used at low concentrations, ProFine™ enhances the efficacy of anti-cancer drugs Docetaxel (a first-line chemotherapy for metastatic castration-resistant prostate cancer) and Enzalutamide (a second-line hormonal therapy for metastatic castration-resistant prostate cancer) (FIG. 3).

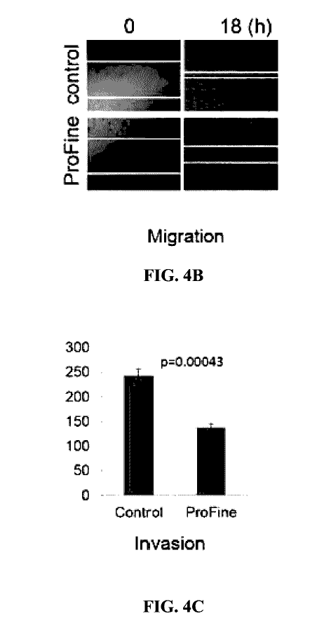

ProFine™ inhibits the invasive behaviors of prostate cancer cells (FIG. 4), suggesting it can potentially reduce metastasis in patients.

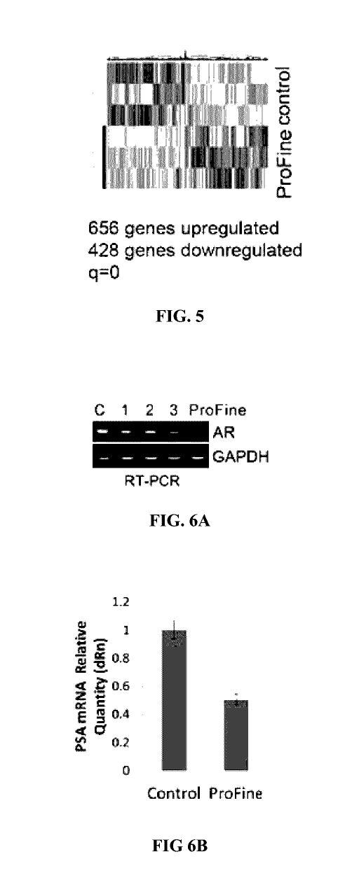

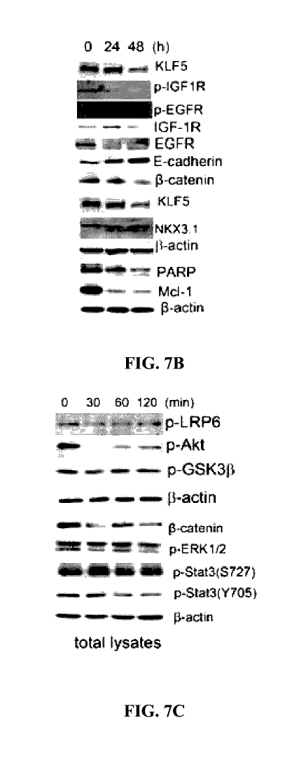

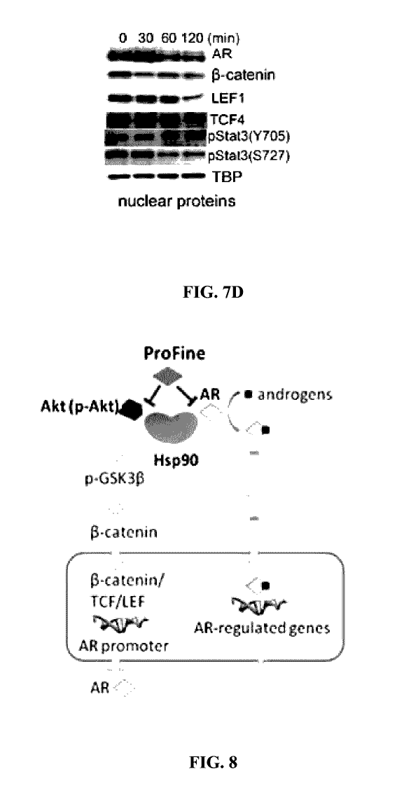

ProFine™ inhibits a wide range of oncogenic genes and signals in metastatic castration-resistant prostate cancer cells (FIG. 5-7, Table 1).

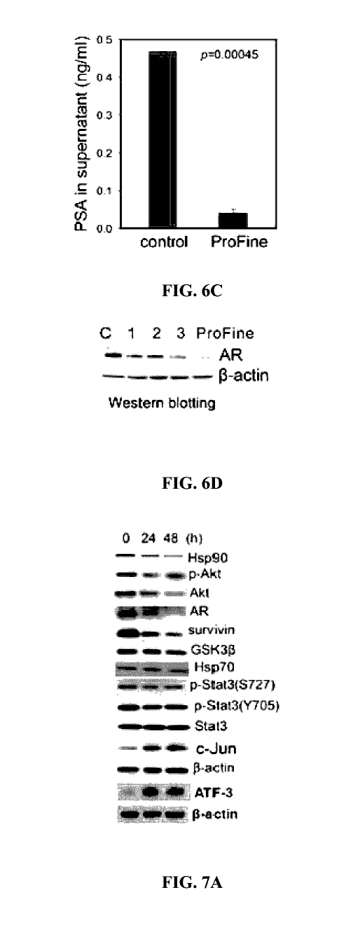

Of particular interest (FIG. 6), ProFine™ inhibits the expression of androgen receptor at both mRNA and protein levels (left), and effectively inhibits the expression (middle) and secretion (right) of prostate-specific antigen (PSA), a clinical indicator of prostate cancer progression.
ProFine™ may inhibit metastatic castration-resistant prostate cancer through a mechanism of action involving the suppression of HSP90, Akt, and androgen receptor signaling (FIG. 8).

### Example 2

### Nutraceutical Composition of ProFine™

Below is an example of pharmaceutical composition of Formula I as a nutraceutical that can be administered via oral route:

### 1.0 ml Final Volume:

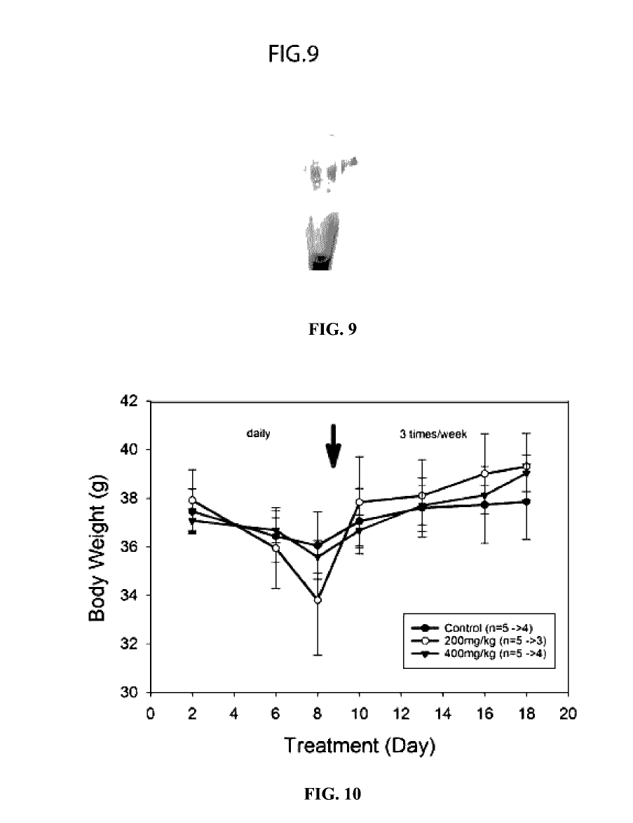

Luteolin: 24.68 mg
Quercetin: 26.06 mg
Kaempferol: 49.35 mg
Hydroxypropyl methylcellulose: 50% (w/v)
Corn oil: 35% (v/v)
Tween 80: 5% (v/v)
Ethanol: 10% (v/v)
   Ultrasonication can be used to form a yellow-colored, well-dispersed colloid formulation (FIG. 9).

### Example 3

### Acute Toxicity of ProFine™ in Rodent Models

The in vivo toxicity of oral ProFine™ was tested in healthy, male CD-1 mice. ProFine™ was prepared as a corn oil-based formulation and administered as two doses (200 mg/kg and 400 mg/kg). A total of 15 CD-1 mice were randomized and divided into three groups (n=5 per group), and given ProFine™ or corn oil (control) via oral gavage, daily for the first week, then three times per week for the second week. Body weights were found to be reduced during the first week, presumably from the stress of oral administration on animals. However, when the schedule of administration changed to three times per week, all mice gained weight (FIG. 10). The behaviors of all mice appeared normal. Blood and plasma samples were collected and analyzed for complete blood count (CBC) and the following chemistry panel (BUN, sodium, potassium, chloride, CO₂, creatinine, glucose, albumin, ALT, ALP, AST, total bilirubin, total protein, calcium, phosphorous, cholesterol). Major organs (liver, lung, spleen, kidney, prostate) were collected. No obvious abnormality was observed. These results indicated ProFine™ is a safe regimen in rodent models, even when administered at high doses up to 400 mg/kg.

### Example 4

### Pharmacokinetics of ProFine™ in Rodent Models

A total of six male Sprague Dawley rats were randomized and evenly divided into two groups (n=3 per group), then administered ProFine™ at the dose of 100 mg/kg via intravenous or oral gavage, respectively. Blood samples were collected at 9 time points, i.e., 0 min, 15 min, 30 min, 60 min, 2 h, 4 h, 8 h, 12 h and 24 h. Plasma levels of Luteolin, Quercetin and Kaempferol were analyzed using HPLC. Pharmacokinetic parameters of the three active ingredients of ProFine™ were described in Table 2.

### Example 5

### In Vivo Efficacy of Oral ProFine™ in Rodent Models

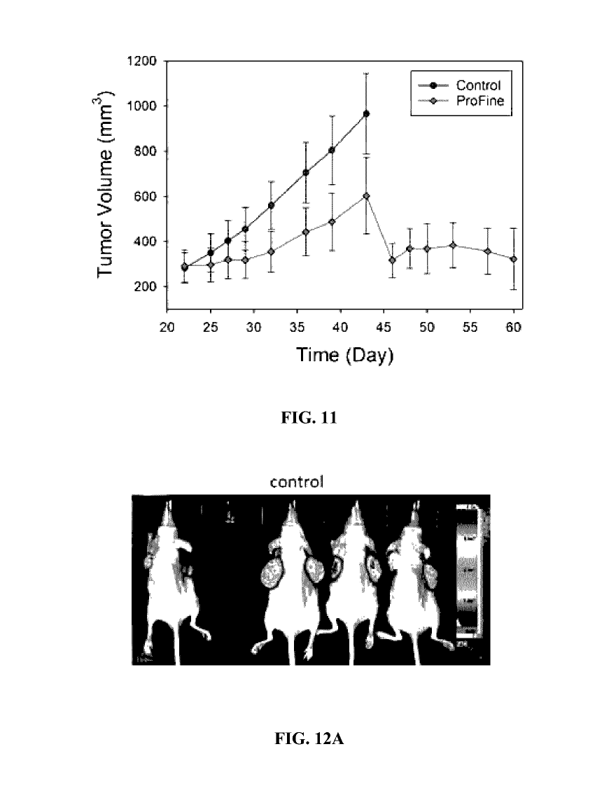

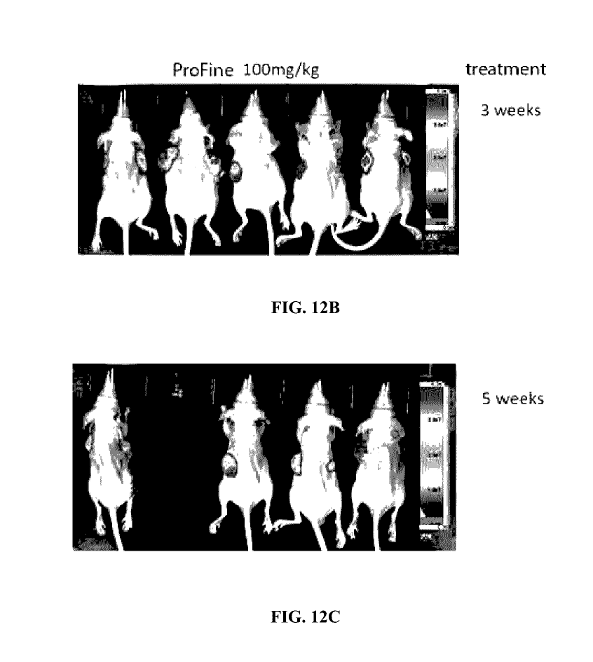

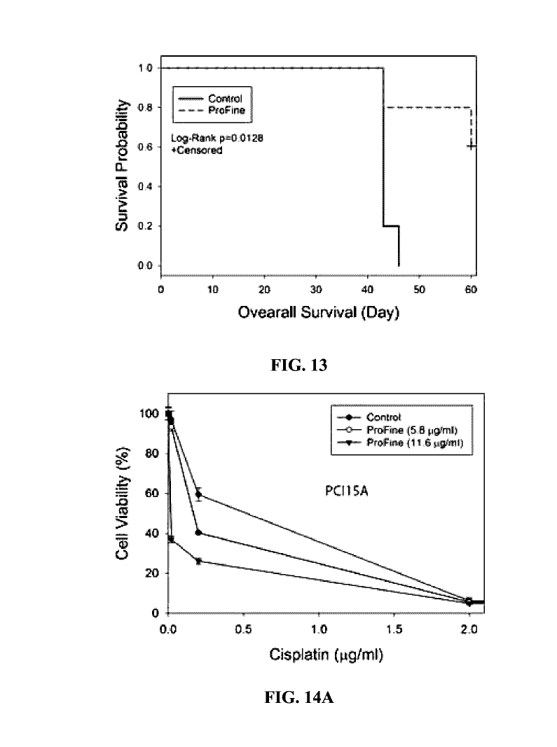

A total of 10 male athymic nude mice (3-4 weeks) were randomized and evenly divided into two groups (n=5 per group). Each mouse was inoculated subcutaneously with 2×10⁶ C4-2-luc prostate cancer cells (mixed with Matrigel) at two sites. Twenty-two days following tumor inoculation, mice were administered with ProFine™ at 100 mg/kg, or control, three times per week, via oral gavage. Tumors were measured three times per week, and tumor volume was calculated using a formula (width)²×length/2. Treatment with ProFine™ reduced tumor burden in mice when compared with control group (FIGS. 11 and 12). Statistical analyses showed that the interaction of time and group is significant (p<0.0001), which means that the groups are changing over time but are changing in different ways, i.e., tumor size in control group increases more quickly than that in ProFine™ group. Although the between-group t-test using a fixed model indicates that it is not significant at day 43 (p=0.254), tumors in ProFine™ group are significantly reduced with treatment time (p<0.001), and two-way ANOVA analysis shows that the ProFine™ treatment results in a significant regression of tumors until day 43 (p=0.0021). Importantly, ProFine™ treatment significantly extended the overall survival of tumor-bearing mice (p=0.0128), as determined by Product-Limit Survival Estimates (FIG. 13).

### Example 6

### Combined Use of ProFine™ with Chemotherapy and Radiation Therapy in Head and Neck Cancer

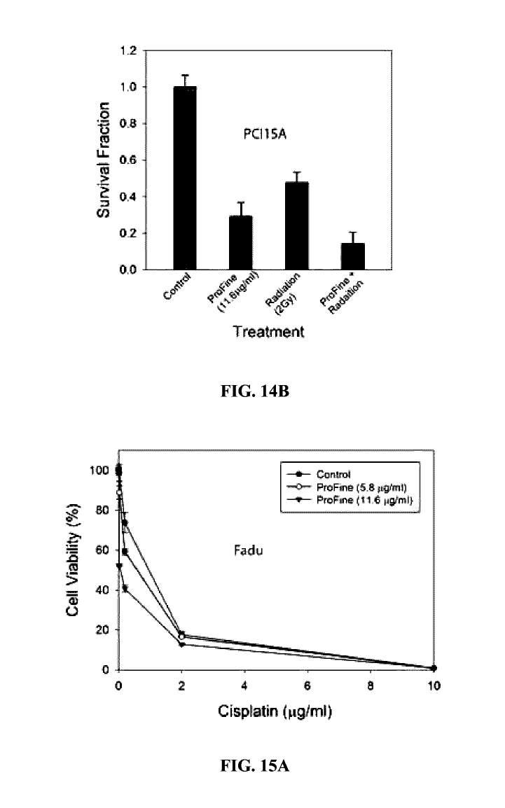

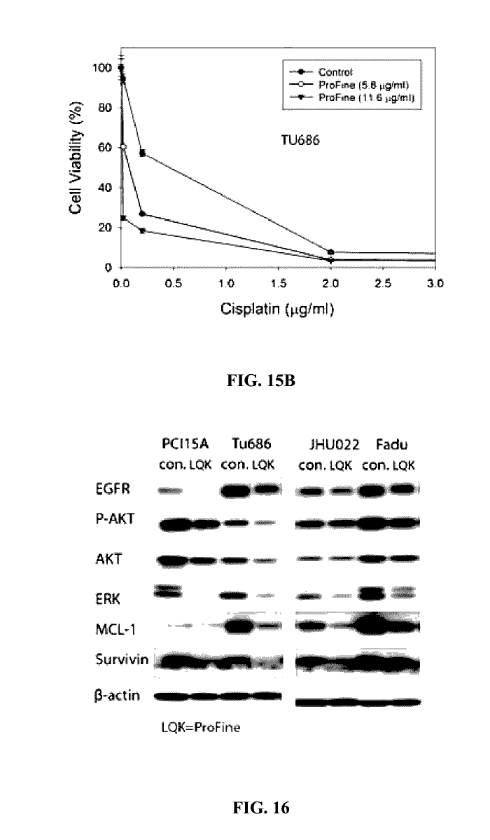

Survival rates in patients with head and neck squamous cell cancer (HNSCC) are about 50%, which have not changed much in the last 50 years, largely due to limited treatment options and therapy-induced toxicities. Resistance to chemoradiation therapy (CRT) remains a major obstacle for the management of locally advanced head and neck squamous cell carcinoma (HNSCC). It is imperative to develop novel strategies to enhance standard therapy and improve clinical outcomes. As shown in FIG. 14 and FIG. 15, ProFine™ enhances the apoptosis-inducing effect of cisplatin and radiation therapy in multiple HNSCC cells. Molecular analysis showed that ProFine™ inhibits several important oncogenic pathways (FIG. 16), including EGFR, p-Akt, p-ERK, Mcl-1 and survivin, which could be responsible for the anti-cancer activity of ProFine™ in HNSCC cells.

TABLE 1
Validated target genes of ProFine
qRT-PCR (C4-2 cells; 5.8 μg/ml, 6 h)

| GeneSymbol | Fold of Change |
|---|---|
| KLK5 | −67.6 |
| NKX3,1 | −24.1 |
| TMPRSS2 | −4.3 |
| PDE9A | −3.3 |
| PMEPA1 | −2.4 |
| PSA | −2.0 |
| CXCL5 | −1.5 |
| ATF3 | +82.2 |
| SLC7A11 | +10.1 |
| VHL | +2.0 |

Table 1 shows a set of validated flavonoid composition target genes in a metastatic castration-resistant prostate cancer cell line (C4-2), as determined by quantitative PCR.

TABLE 2

| Ingredient | 1 | 2 | 3 |
|---|---|---|---|
| Bioavailabilty | 1.30% | 0.91% | 2.89% |
| Cₘₐₓ (ng/ml) | 127.33 | 51.94 | 164.85 |
| Tₘₐₓ (hr) | 1 | 1 | 4 |
| AUC_{0.24} (ng/ml * hr) | 1172.27 (Oral) | 248.15 (Oral) | 2712.51 (Oral) |
| | 90123.12 (IV) | 27204.39 (IV) | 93841.53 (IV) |
| t_{1/2} half-life (hr) | 2.26 | 1.71 | 2.45 |

* ProFine 100 mg/kg (n = 3)

Table 2 shows the pharmacological parameters of flavonoid composition absorption in rat plasma after oral and intravenous administration (1: Luteolin; 2: Quercetin; 3: Kaempferol).
   While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A composition comprising luteolin, quercetin and kaempferol at a molar ratio of 1:1:2.

2. The composition of claim 1, further comprising a pharmaceutically acceptable excipient.

3. The composition of claim 1 formulated for oral administration.

4. The composition of claim 1 formulated for parenteral administration.

5. The composition of claim 1 formulated as a capsule.

6. The composition of claim 1, wherein at least one hydroxyl group of the luteolin, quercetin, or kaempferol is modified to increase water solubility of the composition.

7. The composition of claim 2 wherein the excipient is oil.

8. The composition of claim 7, wherein the oil is vegetable oil.

9. A method of inhibiting or killing cancer cells comprising:
exposing the cancer cells to an inhibitory dose of the composition of claim 1.

10. The method of claim 9, wherein the cancer cells are metastatic castration-resistant prostate cancer cells or head and neck cancer cells.

11. A method for treating a solid tumor in a mammalian subject comprising: administering to the subject a therapeutically effective dose of the composition according to claim 1.

12. The method of claim 11, further comprising the step of repeating the administration at intervals of at least three times per week.

13. The method of claim 12, wherein administration is for a period of at least five weeks.

14. The method of any one of claim 11, wherein the subject is a human subject.

15. The method of claim 9, wherein the composition is administered either as a single regimen or combined with a second cancer treatment regimen.

16. The method of claim 15, wherein the second cancer treatment regimen is selected from the group consisting of hormonal therapy, chemotherapy, and radiotherapy.

17. The method of any one of claim 16, wherein the cancer is prostate cancer or head and neck cancer.

18. The method of claim 9, wherein the composition is administered on a daily basis at a daily dose between 1 to 200 mg/kg body weight.
